(19) United States
(12) Patent Application Publication
(10) Pub. No.: US 2021/0030648 A1
(43) Pub. Date: Feb. 4, 2021

(54) COSMETIC COMPOSITION FOR PRECONDITIONING THE SKIN

(71) Applicant: BEIERSDORF AG, Hamburg (DE)

(72) Inventors: Yanel DE MORAL, Leon (MX); Judith MORAN, Leon (MX); Frank SCHWANKE, Hamburg (DE); Osvaldo Jose Fabian CRUZ CERVANTES, Leon (MX)

(21) Appl. No.: 17/043,032

(22) PCT Filed: Apr. 5, 2018
(86) PCT No.: PCT/EP2018/058661

§ 371 Date: Sep. 29, 2020

(51) Int. Cl. A61K 8/37; A61K 8/81; A61Q 17/04; A61K 8/04; A61K 8/34; A61Q 19/00

(52) U.S. Cl. CPC A61K 8/37; A61K 8/8152; A61Q 17/04; A61K 2800/30; A61K 8/342; A61Q 19/00; A61K 8/042

(57) ABSTRACT

The present invention belongs to the cosmetic field and relates to a cosmetic composition containing a specific combination of an ester, a polymer and water, which allows to increase the contact angle of a droplet of water present on a surface which was preconditioned with the cosmetic composition.

The present invention belongs to the cosmetic field and relates to a cosmetic composition containing a specific combination of an ester, a polymer and water, which allows to increase the contact angle of a droplet of water present on a surface which was preconditioned with the cosmetic composition.

A beautiful and attractive appearance is a desire for many people. One typical sign of such an appearance is a healthy and smooth looking skin. Therefore, in order to take care on the skin, it is for many people a daily routine to apply cosmetic products such as body lotions and sunscreen compositions.

Body lotions generally provide a moisturizing effect and are often used to increase the elasticity and suppleness of the skin. In this way, these products allow the consumer to feel more comfortable in their skin. Sunscreen preparation may not only provide the same beneficial effects such as body lotions. In addition, the sunscreen compositions contain UV-filters to protect the skin from the ultraviolet radiation of the sunlight.

It is well known that in case of an excessive irradiation with light from the UVB range (wavelength: 280-320 nm) acute damage (sunburn) can occur to the skin. Moreover, repetitive exposure to the light from the UVB range may also lead to an increased risk of skin cancer. Furthermore, the radiation from the UVB and UVA (wavelength: 320-400 nm) may also weaken the elastic and collagen fibers of the connective tissue leading to an increased wrinkle formation.

To protect the skin, a number of light protection filter substances have been developed, which can be used in sunscreen compositions. These UVA and UVB filters are in the most developed countries grouped in the form of positive lists such as Annex VI of the Regulation (EC) No 1223/2009 of the European Parliament and of the Council.

However, the wide variety of commercially available sunscreens and body lotions must not obscure the fact that these preparations of the prior art have a number of disadvantages.

In fact, it is often observed that skin preconditioned with a conventional body lotion or sunscreen preparation tends to be completely wetted when being in contact with water. That means a film of water is formed on the skin surface. As a result the water present on the skin may not pearl off that easy and remains on the skin for a prolonged amount of time.

The question whether a film or discrete droplets of water are formed depends very much on the properties of the skin surface. For example, for the cases that water is spilled onto human skin which is not treated or preconditioned with a cosmetic composition it is often found that the water forms discrete droplets with a high contact angle, which do not wetten the entire skin. However, if a droplet of water is spilled on skin preconditioned with a body lotions or sunscreen preparation, the droplet spreads quickly on the skin leading to a low contact angle within a few seconds and a film of water is formed.

The document WO 2011/097448 A1 discloses anhydrous sunscreen formulations containing self-adapting polymers polymerized from a mixture containing either vinylpyrrolidone or vinylacetate. It was found that, if a surface is treated with the compositions disclosed in WO 2011/097448 A1, a droplet of water present on the surface forms a high contact angle with the surface. However, the compositions disclosed in WO 2011/097448 A1 differ from those of the present invention in that they do neither contain the polymers of the present invention nor the specific esters.

Therefore, it remains desirable to provide a cosmetic composition to be applied to the human skin which does not suffer the disadvantages discussed above. In particular, it is the objective of the present invention to provide a cosmetic composition in form of a body lotion or sunscreen preparation, which allows a droplet of water to form a higher contact angle with a surface preconditioned with the cosmetic composition. Thereby, it is also desirable that the skin preconditioned with such a cosmetic composition does not feel oily. Furthermore, it is also desirable that such a cosmetic composition makes the skin feel smooth and the composition is absorbed quickly.

It was surprisingly found that the objective described above can be solved by the present invention.

The invention is a cosmetic composition containing, calculated to the total weight of the composition,
a) at least one ester selected from the group of isodecyl malate, C12-15 alkyl lactate, lauryl lactate and esters according to formula (I)

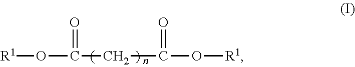

wherein
n is an integer in the range from 2 to 10, preferably in the range from 4 to 8 and most preferably 4; and
R¹ is a linear or branched alkyl group containing 2 to 10 carbon atoms, more preferably 3 to 8 carbon atoms and most preferably 3 to 4 carbon atoms;
b) at least one acrylates/C10-30 alkyl acrylate crosspolymer; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1.

A further object of the present invention is the use of the combination of at least one ester selected from the group of ilsodecyl malate, C12-15 alkyl lactate, lauryl lactate and esters according to formula (I)

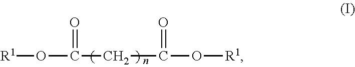

wherein
n is an integer in the range from 2 to 10, preferably in the range from 4 to 8 and most preferably 4; and
R¹ is a linear or branched alkyl group containing 2 to 10 carbon atoms, more preferably 3 to 8 carbon atoms and most preferably 3 to 4 carbon atoms;
and at least one acrylates/C10-30 alkyl acrylate crosspolymer in a cosmetic composition to increase the contact angle of a water droplet present on a surface which was treated or preconditioned with the cosmetic composition.

All the weight percentages (% by weight) given below relate, unless otherwise stated, to the total weight of the cosmetic composition. If ratios of certain components are disclosed in the following description, these ratios refer, unless otherwise stated, to weight ratios of the components.

Unless otherwise stated, all tests and measurements were performed under “normal conditions”. The term “normal conditions” refers to 20° C., 1013 hPa and a relative humidity of 50%.

All disclosed values for the dielectric constant relate to a sample temperature of 25° C. Furthermore, the disclosed values for the dielectric constant relate measurements done at 1013 hPa and a relative humidity of 50%.

For the purposes of the present disclosure, the term “free from” means that the proportion of the respective substance is less than 0.05% by weight. This ensures that entrainments or impurities with these substances are not included as “free from” according to the invention.

According to the invention oils are understood as water immiscible organic substances, which are liquid at 20° C. and 1013 hPa. A substance is understood as water immiscible if less than 100 mg of the substance are dissolvable in 1 l water at 25° C.

The term “skin” refers solely to human skin.

According to the present invention it is preferred if the ester a) is selected from the group of esters according to formula (I) wherein n is an integer in the range from 4 to 8 and more preferably 4, and R¹ is a linear or branched alkyl group containing 3 to 8 carbon atoms and more preferably 3 to 4 carbon atoms.

Thereby it is even more preferred if the ester a) is selected from the group of esters according to formula (I), wherein n=4 and R¹ is an linear or branched alkyl group containing 3 to 4 carbon atoms.

Suitable esters which are according to formula (I) are known under the CTFA declaration dibutyl adipate and diisopropyl adipate.

Furthermore, it is also preferred if the total quantity of the esters a) is in the range from 0.5% to 15% by weight, more preferably 1.5% to 12% by weight and most preferably 2% to 10% by weight, calculated to the total weight of the cosmetic composition.

Furthermore, in one embodiment of the invention it is also preferred if the total quantity of the ester according to formula (I), wherein n is an integer in the range from 4 to 8 and more preferably 4, and R¹ is a linear or branched alkyl group containing 3 to 8 carbon atoms and more preferably 3 to 4 carbon atoms, is in the range from 0.5% to 15% by weight, more preferably 1.5% to 12% by weight and most preferably 2% to 10% by weight, calculated to the total weight of the cosmetic composition.

Still, in another embodiment of the invention it is also preferred if the total quantity of the ester according to formula (I), wherein n=4 and R¹ is a linear or branched alkyl group containing 3 to 4 carbon atoms, is in the range from 0.5% to 15% by weight, more preferably 1.5% to 12% by weight and most preferably 2% to 10% by weight, calculated to the total weight of the cosmetic composition.

Furthermore, the composition of the present invention contains at least one acrylates/C10-30 alkyl acrylate crosspolymer. The at least one acrylates/C10-30 alkyl acrylate crosspolymer b), which is preferably contained in the composition according to the invention, is defined by the fact that, if the polymer is dissolved in a total quantity of 0.2% by weight in water and the resulting mixture is adjusted to a pH of 7.5, the resulting mixture has a viscosity in the range from 500 mPa·s to 8000 mPa·s and more preferably 550 mPa·s to 5500 mPa·s, whereby the viscosity is measured using a Brookfield RV viscometer, spindle RV 4, 20 rpm without Helipath, at 20° C. ambient temperature and 20° C. sample temperature.

According to the invention the pH-adjustment for the measurement of the viscosity of the acrylates/C10-30 alkyl acrylate crosspolymer is either done by the addition of sodium hydroxide or citric acid.

An acrylates/C10-30 alkyl acrylate crosspolymer which is according to the present invention is commercially available under the trade name Pemulen™ EZ-4U sold by the Fa Lubrizol. Viscosity measurements which were made according to invention indicated a viscosity of 4900 mPa·s for Pemulen™ EZ-4U. The acrylates/C10-30 alkyl acrylate crosspolymer which is sold under the trade name Pemulen™ TR-1 by the Fa Lubrizol has a viscosity of 7400 mPa·s, measured as specified above. Pemulen™ TR-2, which is also sold by the Fa Lubrizol, showed a viscosity of 650 mPa·s, measured as specified above.

Furthermore, in terms of the present invention it is preferred if the at least one acrylates/C10-30 alkyl acrylate crosspolymer b) is contained in cosmetic composition in a total quantity in the range from 0.1% to 1% by weight, calculated to the total weight of the composition.

Additionally, preferred cosmetic compositions according to the invention contain water in a total quantity from 60% by weight to 90% by weight and more preferably from 60% by weight to 85% by weight, calculated to the total weight of the composition.

In a first preferred embodiment of the present invention the cosmetic composition contains, calculated to the total weight of the composition,
a) at least one ester selected from the group of esters according to formula (I)

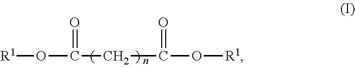

wherein
n is an integer in the range from 4 to 8 and more preferably 4; and
R¹ is a linear or branched alkyl group containing 3 to 8 carbon atoms and more preferably 3 to 4 carbon atoms;
b) at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 500 mPa·s to 8000 mPa·s and more preferably 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1.

Within the first preferred embodiment of the present invention it is more preferred, if the cosmetic composition contains, calculated to the total weight of the composition,
a) at least one ester selected from the group of esters according to formula (I)

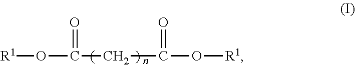

wherein
n is 4; and
R¹ is a linear or branched alkyl group containing 3 to 4 carbon atoms;
b) at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1.

Within the first preferred embodiment of the present invention it is most preferred, if the cosmetic composition contains, calculated to the total weight of the composition,
a) 1.5% to 12% by weight and preferably 2% to 10% by weight of at least one ester selected from the group of esters according to formula (I)

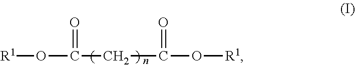

wherein
n is 4; and
R¹ is a linear or branched alkyl group containing 3 to 4 carbon atoms;
b) 0.1% to 1% by weight of at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1.

In a second preferred embodiment of the present invention the cosmetic composition contains, calculated to the total weight of the composition,
a) at least one ester selected from the group of esters according to formula (I)

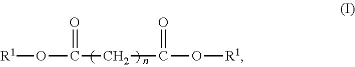

wherein
n is an integer in the range from 4 to 8 and more preferably 4; and
R¹ is a linear or branched alkyl group containing 3 to 8 carbon atoms and more preferably 3 to 4 carbon atoms;
b) at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 500 mPa·s to 8000 mPa·s and more preferably 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1;
   and
   wherein the total quantity of acrylates/C10-30 alkyl acrylate crosspolymers, which are defined by the fact that, if the polymer is dissolved in a total quantity of 0.2% by weight in water and the resulting mixture is adjusted to a pH of 7.5, the resulting mixture has a viscosity of less than 500 mPa·s and/or more than 8000 mPa·s, whereby the viscosity is measured using a Brookfield RV viscometer, spindle RV 4, 20 rpm without Helipath, at 20° C. ambient temperature and 20° C. sample temperature, is less than 0.05% by weight and more preferably 0% by weight, calculated to the total weight of the cosmetic composition.

Within the second preferred embodiment of the present invention it is more preferred, if the cosmetic composition contains, calculated to the total weight of the composition,
a) at least one ester selected from the group of esters according to formula (I)

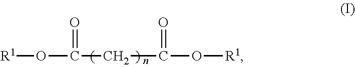

wherein
n is 4; and
R¹ is a linear or branched alkyl group containing 3 to 4 carbon atoms;
b) at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1;
   and
   wherein the total quantity of acrylates/C10-30 alkyl acrylate crosspolymers, which are defined by the fact that, if the polymer is dissolved in a total quantity of 0.2% by weight in water and the resulting mixture is adjusted to a pH of 7.5, the resulting mixture has a viscosity of less than 550 mPa·s and/or more than 5500 mPa·s, whereby the viscosity is measured using a Brookfield RV viscometer, spindle RV 4, 20 rpm without Helipath, at 20° C. ambient temperature and 20° C. sample temperature, is less than 0.05% by weight and more preferably 0% by weight, calculated to the total weight of the cosmetic composition.

Within the second preferred embodiment of the present invention it is most preferred, if the cosmetic composition contains, calculated to the total weight of the composition,
a) 1.5% to 12% by weight and preferably 2% to 10% by weight of at least one ester selected from the group of esters according to formula (I)

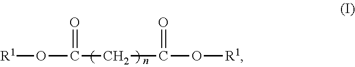

wherein
n is 4; and
R¹ is a linear or branched alkyl group containing 3 to 4 carbon atoms;
b) 0.1% to 1% by weight of at least one acrylates/C10-30 alkyl acrylate crosspolymer, wherein the polymer is defined by the fact that, if the polymer is dissolved in a total quantity of 0.2% by weight in water and the resulting mixture is adjusted to a pH of 7.5, the resulting mixture has a viscosity in the range from 550 mPa·s to 5500 mPa·s, whereby the viscosity is measured using a Brookfield RV viscometer, spindle RV 4, 20 rpm without Helipath, at 20° C. ambient temperature and 20° C. sample temperature; and
c) at least 60% by weight of water;
   wherein the ratio by weight between the esters a) and the acrylates/C10-30 alkyl acrylate crosspolymers b) is in the range from 1:1 to 30:1, more preferably 2:1 to 25:1 and most preferably 3:1 to 18:1;
   and
   wherein the total quantity of acrylates/C10-30 alkyl acrylate crosspolymers, which are defined by the fact that, if the polymer is dissolved in a total quantity of 0.2% by weight in water and the resulting mixture is adjusted to a pH of 7.5, the resulting mixture has a viscosity of less than 550 mPa·s and/or more than 5500 mPa·s, whereby the viscosity is measured using a Brookfield RV viscometer, spindle RV 4, 20 rpm without Helipath, at 20° C. ambient temperature and 20° C. sample temperature, is less than 0.05% by weight and more preferably 0% by weight, calculated to the total weight of the cosmetic composition.

Preferred cosmetic compositions of the present invention are further therein characterized that they contain at least one silicone oil in a total quantity of 0.05% to 2% by weight, more preferably 0.1 to 2% by weight and most preferably 0.2% to 0.5% by weight calculated to the total weight of the composition. According to the invention it is further preferred if the silicone oil contained in the cosmetic composition of the invention is a non-cyclic Polydimethyl-siloxane.

According to the present invention it is further preferred, if the total quantity of all non-silicone oils, which have a dielectric constant of less than 4.0 at 25° C. and which are not an organic UV-filter, is less than 1.5% by weight, more preferably less than 0.8% by weight, more preferably less than 0.5% by weight, more preferably less than 0.2% by weight and most preferably less than 0.01% by weight, calculated to the total weight of the composition.

According to the present invention the dielectric constant of an oil can be determined using dielectric constant meter, e.g. BI-870 Dielectric Constant Meter from the Fa. Brookhaven Instruments Corporation, USA.

The following table indicates the dielectric constants of several organic substances including a number of ester oils. The dielectric constants were determined at a sample temperature of 25° C.

TABLE 1

| INCI | Dielectric constant |
|---|---|
| C12-15 Alkyl Benzoate | 3.8 |
| Isopropyl Myristate | 3.2 |
| Ethylhexyl Palmitate | 3.1 |
| Ethylhexyl Stearate | 3.1 |
| Isocetyl Stearate | 3.0 |
| Octyldodecyl Stearate | 2.8 |
| Mineral Oil | 2.1 |

It is further especially preferred, if the total quantity of C12-15 alkyl benzoate, mineral oil, caprylic/capric triglyceride and/or ethylhexyl palmitate in the cosmetic composition is less than 0.2% by weight and more preferably 0% by weight, calculated to the total weight of the composition.

In general it is the case that cosmetic compositions are preferred which are characterized in that the lipid phase of the composition has a total quantity of 0.5% to 20% by weight, calculated to the total weight of the composition.

According to the invention, it is further preferred if the cosmetic composition contains phenoxyethanol. In the case the cosmetic composition contains phenoxyethanol the total quantity of phenoxyethanol is preferably in the range from 0.1% by weight to 2% by weight and more preferably from 0.4% by weight to 1% by weight, calculated to the total weight of the composition.

Moreover, preferred cosmetic compositions of the present invention are therein characterized that they contain ethylhexylglycerin, whereby it is further preferred if the total quantity of ethylhexylglycerin is in the range from 0.05% to 0.5% by weight, calculated to the total weight of the composition.

Furthermore, it is preferred if the composition does not contain an alkylparabene such as methyl parabene and/or butyl parabene.

Generally, it is further preferred if the total quantity of any further polymeric rheology modifier, in particular polysaccharides, chemically modified polysaccharides, carbomers, sodium polyacrylate and/or further polymers which are polymerized from a mixture containing acrylic acid, methacrylic acid, vinylpyrrolidone and/or vinyl acetate, is less than 0.2% by weight, more preferably less than 0.1% by weight and most preferably 0% by weight, calculated to the total weight of the composition. That means it most preferred if the cosmetic composition does not contain a rheology modifier selected from the group of polysaccharides, chemically modified polysaccharides, carbomers, sodium polyacrylate and/or further polymers which are polymerized from a mixture containing acrylic acid, methacrylic acid, vinylpyrrolidone and/or vinyl acetate.

Moreover, it is preferred according to the present invention if the cosmetic composition contains polymers which are polymerized from a mixture containing polyols and di- or poly-isocyanate in a total quantity of 0% by weight, calculated to the total weight of the composition.

Further preferred cosmetic compositions according to the invention contain glycerol in a total quantity from 1% to 10% by weight, more preferably from 2% to 8% by weight calculated to the total weight of the composition.

Furthermore, preferred embodiments of the present invention are therein characterized that the cosmetic composition contains at least one organic UV-filter.

According to the invention organic UV-filters are those organic compounds which are known from the list of UV filters allowed in cosmetic products from Annex VI of the Regulation (EC) No 1223/2009 of the European Parliament and of the Council, including all amendments and corrections made until the 1^{st} of November 2017. The Regulation (EC) No 1223/2009 of the European Parliament and of the Council, the amendments and corrections can be accessed via the website http://eur-lex.europa.eu/homepage.html.

Organic UV-filter which are preferred according to the invention are selected from the group of compounds comprising camphor benzalkonium methosulfate, homosalate, benzophenone-3, phenylbenzimidazole sulfonic acid, terephthalylidene dicamphor sulfonic Acid, butyl methoxydibenzoylmethane, benzylidene camphor sulfonic acid, octocrylene, polyacrylamidomethyl benzylidene camphor, PEG-PABA, isoamyl P-methoxycinnamate, ethylhexyl triazone, drometrizole trisiloxane, diethylhexyl butamido triazone, 4-methylbenzylidene camphor, ethylhexyl salicylate, ethylhexyl dimethyl PABA, benzohenone-4, benzophenone-5, methylene bis-benzotriazolyl tetramethylbutylphenol, disodium phenyl dibenzimidazole tetrasulfonate, bis-ethylhexyloxyphenol methoxyphenyl triazine, polysilicone-15, diethylamino hydroxybenzoyl hexyl benzoate and tris-biphenyl triazine. Organic UV-filters which are more preferred are selected from the group of compounds comprising homosalate, phenylbenzimidazole sulfonic acid, butyl methoxydibenzoylmethane, octocrylene, ethylhexyl triazone, ethylhexyl salicylate, benzohenone-4, bis-ethylhexyloxyphenol methoxyphenyl triazine and polysilicone-15.

The organic UV-filters which are most preferred according to the invention are selected from the group bis-ethylhexyloxyphenol methoxyphenyl triazine, homosalate, ethylhexyl salicylate, octocrylene and butyl methoxydibenzoylmethane.

According to the invention it is preferred if the total quantity of the organic UV-filters contained in the cosmetic composition is in the range from 1% by weight to 35% by weight, more preferably 8% by weight to 30% by weight and most preferably 10% by weight to 28% by weight, calculated to the total weight of the composition.

In a preferred case of the invention the cosmetic composition contains at least one organic UV-filter selected from the group of compounds comprising bis-ethylhexyloxyphenol methoxyphenyl triazine, homosalate, ethylhexyl salicylate, octocrylene and butyl methoxydibenzoylmethane, whereby the total quantity of the organic UV-filters selected from the group of compounds comprising bis-ethylhexyloxyphenol methoxyphenyl triazine, homosalate, ethylhexyl salicylate, octocrylene and butyl methoxydibenzoylmethane is in the range from 1% by weight to 35% by weight, more preferably 8% by weight to 30% by weight and most preferably 10% by weight to 28% by weight, calculated to the total weight of the composition.

In the case that the cosmetic composition of the present invention contains bis-ethylhexyloxyphenol methoxyphenyl triazine it is preferred if the total quantity of bis-ethylhexyloxyphenol methoxyphenyl triazine is in the range from 0.1% by weight to 5% by weight, more preferably from 0.5% by weight to 4.8% by weight and most preferably from 1% by weight to 4.5% by weight, calculated to the total weight of the composition.

In the case that the cosmetic composition of the present invention contains homosalate it is preferred if the total quantity of homosalate is in the range from 1% by weight to 15% by weight, more preferably from 3% by weight to 10% by weight and most preferably from 5% by weight to 8% by weight, calculated to the total weight of the composition.

In the case that the cosmetic composition of the present invention contains ethylhexyl salicylate it is preferred if the total quantity of ethylhexyl salicylate is in the range from 1% by weight to 12% by weight, more preferably from 2% by weight to 8% by weight and most preferably from 3% by weight to 6% by weight, calculated to the total weight of the composition.

In the case that the cosmetic composition of the present invention contains octocrylene it is preferred if the total quantity of octocrylene is in the range from 0.1% by weight to 10% by weight, more preferably from 1% by weight to 9% by weight and most preferably from 2% by weight to 8% by weight, calculated to the total weight of the composition.

In the case that the cosmetic composition of the present invention contains butyl methoxydibenzoylmethane it is preferred if the total quantity of butyl methoxydibenzoylmethane is in the range from 0.5% by weight to 10% by weight, more preferably from 1% by weight to 8% by weight and most preferably from 3% by weight to 5% by weight, calculated to the total weight of the composition.

Furthermore, preferred compositions according to the invention are therein characterized that the compositions do not contain ethylhexyl methoxycinnamate.

Moreover, the cosmetic compositions of the present invention are preferably free from inorganic UV-filter, especially free from zinc oxide and titanium dioxide. It is especially preferred, if the total quantity of zinc oxide and titanium dioxide in the cosmetic composition of the present invention is less than 0.1% by weight and more preferably 0% by weight, calculated to the total weight of the composition.

Preferred embodiments of the invention are also therein characterized that the composition is a purely liquid composition, meaning that the composition does not contain any particulate matter which is solid at a temperature above 90° C. at 1013 hPa.

Furthermore, it was noted that preferred cosmetic compositions according to the invention are therein characterized that the cosmetic composition contains a gel structure, preferably a micro-gel structure.

In addition, preferred cosmetic compositions are characterized in that they contain ethanol in a total quantity of less than 0.1% by weight and more preferably 0% by weight, calculated to the total weight of the composition.

Furthermore, it was also surprisingly noticed that the addition of cetyl alcohol and/or stearyl alcohol reduces tackiness of the cosmetic composition during application. The addition of behenyl alcohol did not reduce the tackiness during application. Therefore, it is further preferred, if the cosmetic composition contains cetyl alcohol and/or stearyl alcohol. Mixtures of cetyl alcohol and stearyl alcohol can be obtained under the INCI designation cetearyl alcohol. If the composition contains cetyl alcohol and/or stearyl alcohol, it is further preferred, if the total quantity of cetyl alcohol and/or stearyl alcohol is in the range from 0.2% to 3% by weight, more preferably 0.5% to 2% by weight, calculated to the total weight of the composition.

According to the invention, the cosmetic composition is advantageously further therein characterized that the composition contains any further emulsifier or a surfactant in a total quantity of less than 2% by weight, more preferably less than 1% by weight, more preferably less than 0.5% by weight, more preferably 0.2% by weight and most preferably 0% by weight, calculated to the total weight of the composition. There term “further emulsifier” is directed to the fact that in some instances the polymer acrylates/C10-30 alkyl acrylate crosspolymer is described as polymeric emulsifier.

According to the invention the cosmetic composition may preferably contain at least one compound selected from the group of limonene, linalool, citral, alpha-isomethylionone and geraniol.

Embodiments of the present invention which are advantageous according to the invention are characterized in that the cosmetic composition contains as further ingredients one or more compounds selected from the group of the compounds containing alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, tocopheryl acetate, dihydroxyacetone, glycyrrhetinic acid, 8-hexadecene-1, 16-dicarboxylic acid, glycerylglycose, (2-hydroxyethyl) urea and/or licochalcone A.

Moreover, it is further preferred if the cosmetic composition according to the invention has a pH value in the range from 4.5 to 8.

Additionally, preferred cosmetic compositions of the present invention are therein characterized that they have a viscosity in the range from 1500 mPa·s to 8000 mPa·s, more preferably 3500 mPa·s to 7000 mPa·s, whereby the viscosity is measured using a Brookfield RV viscometer, spindle RV 4, 20 rpm without Helipath, at 20° C. ambient temperature and 20° C. sample temperature.

Generally, the cosmetic compositions according to the present invention can be prepared by common formulation techniques for emulsions. However, in the case the cosmetic composition according to the invention contains an UV-filter which is soluble in water it may happen that the water-soluble UV-filter affects the gel formation of the acrylates/C10-30 alkyl acrylate crosspolymer resulting in a loss of viscosity. It was surprisingly found that this problem can be addressed by adding the water-soluble UV-filter after the mixing of all other ingredients. This means the water-soluble UV-filter is preferably added after the formation of the gel structure.

### EXAMPLES

The following examples should illustrate the compositions of this invention, without intending to limit the invention to these examples. The numerical values in the examples are percentages by weight, based on the total weight of the preparations.

In a first study a visual assessment was performed analyzing what shape a water droplet forms on the skin preconditioned with the formulations presented in the following table:

TABLE 2

| INCI | Inv. 1 | Inv. 2 | Inv. 3 | Com. 1 |
|---|---|---|---|---|
| Glyceryl Glucoside | 0.004 | 0 | 0.004 | 0 |
| Ethylhexylglycerin | 0.15 | 0 | 0.15 | 0.5 |
| Tocopheryl Acetate | 0 | 0 | 0 | 0.06 |
| Glycyrrhetinic Acid | 0 | 0 | 0 | 0.1 |
| Diisopropyl Adipate | 8 | 3 | 8 | 0 |
| Butylene Glycol | 1 | 0 | 1 | 3 |
| Dicaprylate/Dicaprate | | | | |
| Dimethicone | 0.3 | 0.3 | 0.3 | 0 |
| C18-36 Acid Triglyceride | 0 | 0 | 0 | 0.5 |
| Ceteareth-20 | 0 | 0 | 0 | 1 |
| VP/Hexadecene Copolymer | 0 | 0 | 0 | 0.5 |
| Parfum | 0 | 0 | 0 | q.s. |
| Glycerin | 6 | 0 | 6 | 8.6 |
| Sodium Hydroxide | q.s. | q.s. | q.s. | q.s. |
| Phenoxyethanol | 0.8 | 0.8 | 0.8 | 0.6 |
| Ethylparaben | 0 | 0 | 0 | 0.1 |
| Methylparaben | 0 | 0 | 0 | 0.3 |
| Carbomer | 0.16 | 0 | 0.16 | 0 |
| Acrylates/C10-30 Alkyl | 0.5 | 0.2 | 0 | 0 |
| Acrylate Crosspolymer | | | | |
| (Pemulen EZ-4U, Lubrizol) | | | | |
| Acrylates/C10-30 Alkyl | 0 | 0 | 0.5 | 0 |
| Acrylate Crosspolymer | | | | |
| (Pemulen TR-2, Lubrizol) | | | | |
| Acrylates/C10-30 Alkyl | 0 | 0 | 0 | 0.2 |
| Acrylate Crosspolymer | | | | |
| (Pemulen TR-1, Lubrizol) | | | | |
| Acrylates/C10-30 Alkyl | 0 | 0 | 0 | 0.2 |
| Acrylate Crosspolymer | | | | |
| (Carbopol 1382) | | | | |
| Alcohol Denat. | 0 | 0 | 0 | 5.75 |
| Trisodium EDTA | 0 | 0 | 0 | 0.2 |
| Homosalate | 0 | 8 | 0 | 9 |
| Octocrylene | 0 | 2 | 0 | 8 |
| Ethylhexyl Salicylate | 0 | 4.5 | 0 | 4.5 |
| Bis-Ethylhexyloxyphenol | 0 | 1 | 0 | 3.5 |
| Methoxyphenyl Triazine | | | | |
| Butyl | 0 | 3.5 | 0 | 4.5 |
| Methoxydibenzoylmethane | | | | |
| Phenylbenzimidazole | 0 | 0 | 0 | 1 |
| Sulfonic Acid | | | | |
| Aqua | ad 100 | ad 100 | ad 100 | ad 100 |

The visual assessment study was carried out in the following manner.
1. A section of 5 cm diameter was marked on the clean skin of a forearm of the participants.
2. 25 μl of each of the compositions listed in table 2 were applied to a separate section and evenly distributed over the marked area with a fingertip.
3. It was waited for 5 minutes to let the composition absorb.
4. A water droplet of 25 μl was then placed on the marked section. Furthermore, outside of the treated and marked section further water droplets were placed as reference.
5. A photograph was taken of the forearm within 20 seconds after application and the shape of the water droplets was analyzed.

It was found that water droplets placed on the sections with the inventive compositions Inv. 1, Inv. 2 and Inv. 3 showed a similar high contact angle such as the water droplet placed on the non-treated section. In contrast to that the water droplet placed on the section treated with the non-inventive composition Com. 1 spread out and showed therefore a low contact angle.

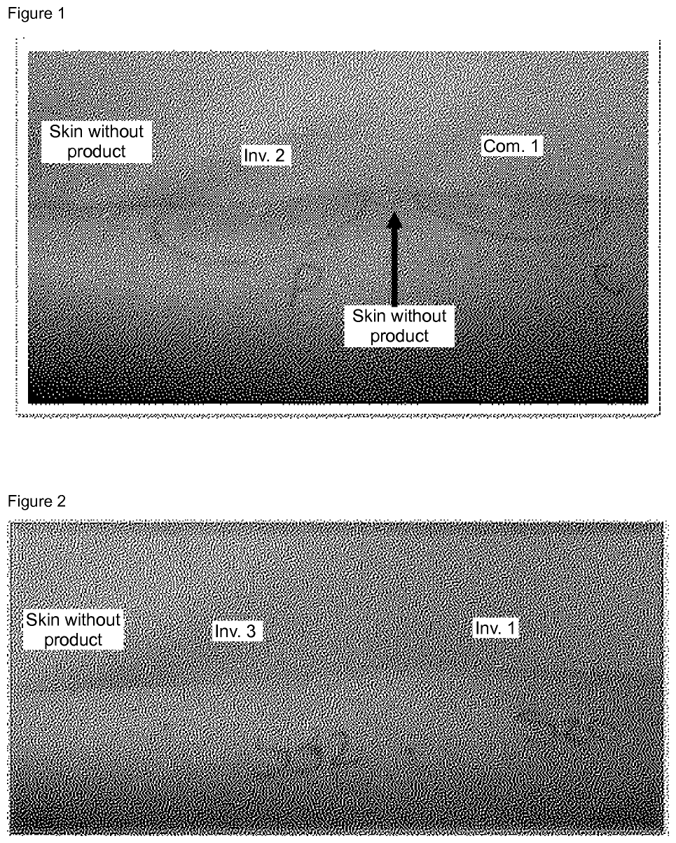

FIG. 1 shows a photography of the water droplets placed on the sections treated with the compositions Inv. 2 and Com. 1. Furthermore, water droplets placed on non-treated sections are shown.

FIG. 2 shows a photography of the water droplets placed on the sections treated with the compositions Inv. 1 and Inv. 3. Furthermore, water droplets placed on non-treated sections are shown.

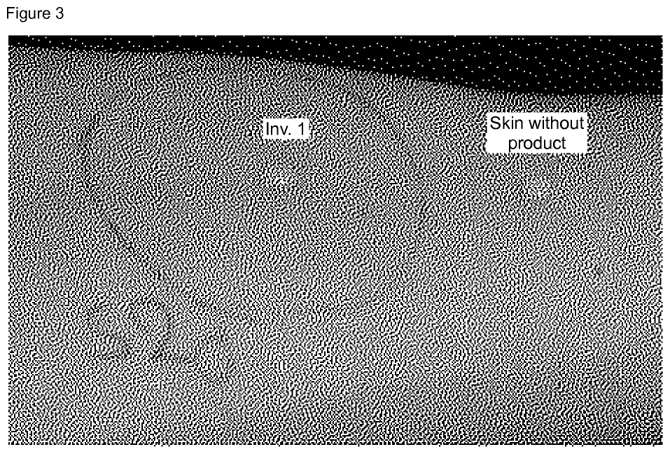

FIG. 3 shows a further photography of a water droplet placed on the section treated with the compositions Inv. 1. Furthermore, a water droplet placed on non-treated sections is shown.

In a further study the contact angle of a water droplet placed on a surface, which was treated with the compositions Inv. 1, Inv. 2 or Com. 1, was measured. The measurement of the contact angle was carried out in the following manner:
1. 1.3 mg/cm² of the sample formulas were placed on a PMMA plate (sandblasted PMMA-Plates type WW5 (RA=5 μm roughness), Schonberg Acrylic Design in Hamburg, Schnelsen)
2. The samples were spread with a finger cover for 10 times with circling motions
3. It was waited for 20 to 30 minutes to let the composition dry.
4. The plates with samples were either non treated or treated with 30 μl of a phosphate buffer solution (PBS), a protein solution (containing Bovine serum albumin) or demineralized water. Then it was waited until the samples were dry.
5. A droplet of water was placed on each plate and two measurements of the contact angle were performed using a goniometer (Dataphysics OCA 20). The first measurement was performed immediately when the droplet was placed on the sample plate, while the second measurement was performed 2 seconds after the drop of water had flattened out.

The experimental results are presented in table 3, wherein the first value corresponds to the immediate measurement and the second value to the measurement performed 2 seconds after the drop of water had flattened out:

TABLE 3

| Formula | No further treatment | PBS solution | Protein solution | Demineralized water |
|---|---|---|---|---|
| Com. 1 | 40.96°/24.03° | 41.17°/11.19° | 32.52°/11.37° | 47.82°/13.83° |
| Inv. 1 | 85.4°/52.43° | 68.51°/54.22° | 82.75°/54.87° | 84.71°/53.16° |
| Inv. 2 | 79.12°/65.65° | 76.19°/63.49° | 81.03°/70.19° | 79.63°/70.39° |

It can be noted from the results that the inventive compositions Inv. 1 and Inv. 2 both show a higher contact angle for the droplet of water than the non-inventive composition Com. 1. The results are in agreement with the visual analysis described above.

The following examples shall further exemplify the present invention.

| INCI | Ex. 1 | Ex. 2 | Ex. 3 | Ex. 4 | Ex. 5 |
|---|---|---|---|---|---|
| Glyceryl Glucoside | 0.004 | 0 | 0.004 | 0.004 | 0.004 |
| Ethylhexylglycerin | 0.15 | 0 | 0.15 | 0.15 | 0.15 |
| Diisopropyl Adipate | 8.5 | 3.5 | 7 | 8 | 8 |
| Butylene Glycol | 1 | 0 | 0.5 | 1 | 1 |
| Dicaprylate/Dicaprate | | | | | |
| Dimethicone | 0.3 | 0.3 | 0.3 | 0.3 | 0.3 |
| Cetearyl Alcohol | 0 | 0 | 0 | 0 | 1 |
| Parfum | 0 | 0 | 0 | q.s. | q.s. |
| Glycerin | 6 | 0 | 6 | 6 | 6 |
| Sodium Hydroxide | q.s. | q.s. | q.s. | q.s. | q.s. |
| Phenoxyethanol | 0.8 | 0.8 | 0.8 | 0.8 | 0.8 |
| Ethylparaben | 0 | 0 | 0 | 0 | 0 |
| Methylparaben | 0 | 0 | 0 | 0 | 0 |
| Carbomer | 0.16 | 0 | 0.16 | 0.16 | 0.16 |
| Acrylates/C10-30 Alkyl | 0.6 | 0.15 | 0 | 0 | 0 |
| Acrylate Crosspolymer | | | | | |
| (Pemulen EZ-4U, | | | | | |
| Lubrizol) | | | | | |
| Acrylates/C10-30 Alkyl | 0 | 0.15 | 0.4 | 0 | 0 |
| Acrylate Crosspolymer | | | | | |
| (Pemulen TR-2, | | | | | |
| Lubrizol) | | | | | |
| Acrylates/C10-30 Alkyl | 0 | 0 | 0 | 0.5 | 0.5 |
| Acrylate Crosspolymer | | | | | |
| (Pemulen TR-1, | | | | | |
| Lubrizol) | | | | | |
| Alcohol Denat. | 0 | 0 | 0 | 5.75 | 5.75 |
| Trisodium EDTA | 0 | 0 | 0 | 0 | 0 |
| Homosalate | 0 | 8 | 9 | 0 | 0 |
| Octocrylene | 0 | 2 | 8 | 0 | 0 |
| Ethylhexyl Salicylate | 0 | 4.5 | 4.5 | 0 | 0 |
| Bis- | 0 | 1 | 3.5 | 0 | 0 |
| Ethylhexyloxyphenol | | | | | |
| Methoxyphenyl Triazine | | | | | |
| Butyl Methoxy- | 0 | 3.5 | 4.5 | 0 | 0 |
| dibenzoylmethane | | | | | |
| Phenylbenzimidazole | 0 | 0 | 1 | 0 | 0 |
| Sulfonic Acid | | | | | |
| Aqua | ad 100 | ad 100 | ad 100 | ad 100 | ad 100 |

1.-22. (canceled)

23. A cosmetic composition, wherein the composition comprises (a) at least one ester selected from isodecyl malate, C₁₂₋₁₅ alkyl lactate, lauryl lactate and esters of formula (I) 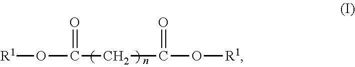
wherein
n is an integer of 2 to 10 and R¹ is a linear or branched alkyl group having from 2 to 10 carbon atoms;
(b) at least one acrylates/C10-30 alkyl acrylate crosspolymer; and
(c) at least 60% by weight of water, based on a total weight of the composition;
the ratio by weight of (a) and (b) ranging from 1:1 to 30:1.

24. The composition of claim 23, wherein (a) is selected from esters of formula (I) wherein n is an integer of from 4 to 8 and Rⁱ is a linear or branched alkyl group having from 3 to 8 carbon atoms.

25. The composition of claim 23, wherein (a) is selected from esters of formula (I), wherein n=4 and R¹ is a linear or branched alkyl group having 3 or 4 carbon atoms.

26. The composition of claim 23, wherein the composition comprises from 0.5% to 15% by weight of (a), based on the total weight of the composition.

27. The composition of claim 23, wherein if (b) is dissolved in a total quantity of 0.2% by weight in water and a resulting mixture is adjusted to a pH of 7.5, the resulting mixture has a viscosity of from 500 mPa-s to 8000 mPa-s, as measured using a Brookfield RV viscometer, spindle RV 4, 20 rpm without Helipath, at 20° C. ambient temperature and 20° C. sample temperature.

28. The composition of claim 27, wherein the resultant mixture has a viscosity of from 550 mPa-s to 5500 mPa-s.

29. The composition of claim 23, wherein the composition comprises (b) in a total concentration of from 0.1% to 1% by weight, based on the total weight of the composition.

30. The composition of claim 23, wherein the composition comprises from 60% to 90% by weight of (c).

31. The composition of claim 23, wherein the composition further comprises from 0.05% to 2% by weight, based on the total weight of the composition, of at least one silicone oil.

32. The composition of claim 23, wherein the composition comprises non-silicone oils which have a dielectric constant of less than 4.0 at 25° C. and are not organic UV filters in a total concentration of from 0% to less than 0.5% by weight, based on the total weight of the composition.

33. The composition of claim 23, wherein a total concentration of C₁₂₋₁₅ alkyl benzoate, mineral oil, caprylic/capric triglyceride and ethylhexyl palmitate is from 0% to less than 0.2% by weight, based on the total weight of the composition.

34. The composition of claim 23, wherein the composition comprises from 0.5% to 20% by weight of a lipid phase, based on the total weight of the composition.

35. The composition of claim 23, wherein the composition comprises polysaccharides, chemically modified polysaccharides, carbomers, sodium polyacrylate and/or further polymers which are polymerized from a mixture comprising acrylic acid, methacrylic acid, vinylpyrrolidone and/or vinyl acetate in a total concentration of from 0% to less than 0.2% by weight, based on the total weight of the composition.

36. The composition of claim 23, wherein the composition is free of polymers which are polymerized from a mixture containing polyols and di- or poly-isocyanates.

37. The composition of claim 23, wherein the composition comprises at least one organic UV filter.

38. The composition of claim 37, wherein the at least one organic UV filter is selected from bis-ethylhexyloxyphenol methoxyphenyl triazine, homosalate, ethylhexyl salicylate, octocrylene, and butyl methoxydibenzoylmethane and a total concentration of the at least one organic UV filter selected from bis-ethylhexyloxyphenol methoxyphenyl triazine, homosalate, ethylhexyl salicylate, octocrylene, and butyl mthoxydibenzoylmethane is from 1% by weight to 35% by weight, based on the total weight of the composition.

39. The composition of claim 23, wherein the composition comprises a gel structure.

40. The composition of claim 23, wherein the composition further comprises from 0% to less than 0.1% by weight of ethanol, based on the total weight of the composition.

41. The composition of claim 23, wherein the composition has a viscosity of from 1500 mPa-s to 8000 mPa-s, as measured using a Brookfield RV viscometer, spindle RV 4, 20 rpm without Helipath, at 20° C. ambient temperature and 20° C. sample temperature.

42. The composition of claim 23, wherein the composition further comprises from 0.2% to 3% by weight of cetyl alcohol and/or stearyl alcohol, based on the total weight of the composition.
